(19) United States
(12) Patent Application Publication
(10) Pub. No.: US 2004/0023374 A1
(43) Pub. Date: Feb. 5, 2004

(54) Method and apparatus for multi-layer growth of anchorage dependent cells

(76) Inventors: Catherine Rappaport, Salt Lake City, UT (US); Edward Trujillo, Salt Lake City, UT (US); Yvonne Rensch, Salt Lake City, UT (US); Masoud Abbasi, St. Paul, MN (US); Michael Kempe, Salt Lake City, UT (US); Christian Rocaboy, Erlangen (DE); John Gladysz, Salt Lake City, UT (US)

Correspondence Address: Madson & Metcalf, 15 West South Temple, Suite 900, Salt Lake City, UT, 84101, US

(21) Appl. No.: 10/416,561

(22) PCT Filed: Nov. 13, 2001
(86) PCT No.: PCT/US01/47061

(51) Int. Cl. C12N 5/06; C12M 1/14; C12M 3/04

(52) U.S. Cl. 435/325; 435/299.1

(57) ABSTRACT

Anchorage-dependent cells are grown in a novel cell culture plate and on a novel substratum which increase the oxygenation of the cells. The cell culture plate is made by enclosing a growth chamber within a shell made of a solid sterilizable. One or more culture wells are positioned within the chamber. An inlet port and outlet port are fashioned within the shell for gas exchange. The wells have a well wall which allows for the diffusion of oxygen from the chamber into the well. A perfluorocarbon is placed within the well. A perfluoro-aldehyde is mixed with the perfluorocarbon, and the perfluoro-aldehyde re-orients so that the aldehyde head groups are at the interface. An attachment factor is bound to the perfluoro-aldehyde, which is sunk into the PFC substratum. Aqueous growth media is then added to the well, and anchorage-dependent cells added and allowed to grow.

### 1. FIELD OF THE INVENTION

The present invention relates to systems and methods for cultivating cells. More particularly, the present invention relates to a system and method for supporting multilayer growth of anchorage-dependent cells.

### 2. TECHNICAL BACKGROUND

Cell cultures are cells from a plant or animal which are grown outside the organism from which they originate. These cells are of ten grown, for example, in petri dishes under specific environmental conditions. Cell cultures are of great importance because they represent biological factories capable of producing large quantities of bioproducts such as growth factors, antibodies, viruses, and vaccines. These products can then be isolated from the cell cultures and used, for example, to treat human disease and as vaccines. In addition, tissue cultures are a potential source of tissues and organs which could be used for transplantation into humans. For example, tissue cultured skin cells are being used in skin grafts. Finally, tissue cultures usually comprise cells from only one or a few tissues or organs. Consequently, cell cultures provide scientists a system for studying the properties of individual cell types without the complications of working with the entire organism.

In vivo, cells form complex multilayer structures which ultimately form tissues and organs. These cells receive their nutrient and oxygen requirements via the blood in the circulatory system. In addition, in order to form tissues and organs, cells must form contacts with each other and with an extracellular matrix. Extracellular matrices comprise a complex and variable array of collagens, glycosaminoglycans, proteoglycans, and glycoproteins. Together these cellular products form the basal lamina, bone, and cartilage which give tissues and organs their shape and strength. The contact between anchorage-dependent cells and the extracellular matrix plays a dramatic role in determining the cells' shape, position, metabolism, differentiation and growth.

Like most cells in vivo, many cells are anchorage-dependent; that is, they can metabolize and divide only if they are attached to a surface or substratum. Only cells of the circulatory system (e.g., lymphocytes and red blood cells) grow unattached and suspended in solution in vitro. While many anchorage-dependent cells may grow on glass or plastic surfaces, these cells lose their ability to differentiate and respond to hormones. For this reason, glass and plastic tissue culture dishes are of ten coated with an extracellular matrix such as collagen. Unlike cells in vivo, normal cells in culture do not form significant multilayer structures. Under optimal conditions, for example, epithelial cells grow only one cell layer thick (monolayer), while fibroblast cells at best grow two or three layers thick. Once the growing surface is confluent with cells, normal cells cease to divide and their number actually begins to decline with time. This phenomenon is referred to as “density-dependent inhibition.”

The failure of cells to grow to form multilayered structures is a major limitation of current tissue culture techniques. Cells growing in monolayers lose the capacity to perform many of the essential functions that they perform in their respective tissues and organs. This is primarily due to the fact that in vivo these cells are surrounded by other cells which provide many factors needed for normal function and growth. Thus, current research is hindered by the fact that tissue culture techniques do not accurately mimic in vivo biological activity.

It is believed that the inability of cells in culture to form multilayer structures is owed, in part, to lack of oxygenation. Oxygen, unlike other nutrients, is only sparingly soluble in aqueous media. Thus, cells several cell layers deep do not receive sufficient oxygen to grow and maintain normal biological activity.

Developing methods for improving oxygenation of anchorage-dependent mammalian cells in tissue culture is an active field of investigation. It has long been recognized that growth of cells on the bottom of a culture dish would be limited by the slow diffusion of oxygen from air through the medium. More recent measurements with micro-oxygen electrodes have shown that, with every cell line tested, cells were growing under conditions where the oxygen provided did not support their full respiratory capacity. This could account for the de-differentiation cells undergo in vitro since they could not generate the energy needed to maintain complex functions involved.

Conventionally, two methods have been used to improve oxygenation for cell growth in bioprocesses: mechanical stirring and bubbling. Cultures have been mechanically stirred to increase oxygen transport at the aqueous/air interface and distribute oxygen uniformly in the culture fluid. This results in shear and other problems with anchorage-dependent mammalian cells which lack a rigid cell wall and are large (10-100 μm) and very fragile. Also, anchorage-dependent mammalian cells cannot rotate or translate freely to reduce the net forces and torques from shear because they are attached and fixed to the substratum. Studies suggest that the viability of endothelial and human kidney cells is profoundly affected by shear. These effects are observed at shear rates as low as 1 dyne/cm². Cell shear stress is also created when air bubbles contact the cell or bioparticle. Typically, higher bubbling flow rates per unit volume increase the specific death rate of anchorage-dependent mammalian cells in microcarrier systems.

Shear stress may also cause changes in the shape and function of cells. If the stress is strong enough, anchorage-dependent cells can be detached from the surface to which they are attached. In addition, some cell functions, such as cytoskeleton assembly, metabolism, biomolecular synthesis, are also shear stress dependent, even under conditions of laminar flow.

Many investigators have added a polymer to the medium to reduce hydrodynamic effects. This has been found to provide some protection. The most frequently used polymers are methylcellulose, polysucrose, Dextran, and Pluronic F-68. It is believed that the polymers adhere to the cell surface and form a protective shell against shear and mechanical forces. However, whether a protective shell is actually formed or the effects it may have on normal cell function is unknown.

Another method for increasing oxygenation to cell cultures is the hollow fiber membrane bioreactor. In this system, the cells are attached to a cylindrical hollow fiber membrane. Culture media and oxygen flows through the center of the cylindrical hollow fiber membrane. The molecular weight cut-off of the membrane permits nutrients and oxygen to reach the cells without allowing the cells to escape. However, the cells grown with this method do grow to form multilayers of cells.

Apart from the problem of poor oxygenation, current tissue culture techniques do not address the problem of controlling production of free radicals. Free radicals are atoms or polyatomic molecules which posses one unpaired electron. They can arise in the medium by reaction of oxygen with iron or copper, as well as with some metabolites in the medium. They are also produced as electrons move down the mitochondrial electron transport chain and are known to increase with increased respiration. Since free radicals are toxic to mammalian cells, even at very low levels, the growth benefits derived from increased respiration would depend on providing antioxidants to counter the different type of free radicals which may be produced.

More recently, perfluorocarbons (PFCs) have been used to increase oxygenation of cultures. Perfluorocarbons are organic compounds where all hydrogen atoms are replaced by fluorine atoms. Oxygen is 15 to 20 times more soluble in PFCs that in water. As a result, PFCs are sometimes referred to as psuedoerythrocytes because they are oxygen-carrying molecules analogous to the erythrocytes that carry oxygen in mammalian blood. Indeed, PFCs have been used as oxygen carriers in place of red blood cells in animals.

Current tissue culture techniques which employ PFCs, however, have their own limitations. One method teaches continuously adding an oxygenated PFC to the top of the culture media. The PFC being denser than the aqueous culture media sinks to the bottom of the bioreactor where it is removed. While this system successfully improved the oxygenation of the culture media, it has two significant disadvantages. First, the system is limited to suspension cells which must be mechanically stirred in order to prevent them from settling at the bottom of the reactor. As discussed above, this damages mammalian cells. Second, the PFC comes into direct contact with the cells. PFC has been shown to alter the normal biological activity of various cells in culture. For example, neutrophils and monocytes incubated with PFCs exhibit decreased phagocytic activity, chemotaxis, aggregation, cellular adherence, and superoxide ion release.

Currently, cultivation of anchorage-dependent mammalian cells using PFCs has been unsuccessful. The principal difficulty is that anchorage-dependent mammalian cells do not adhere to PFC surfaces or do not adhere any better than on conventional polystyrene surfaces. A few studies have reported some growth on microspheres (diameter 100 to 500 μm) made by emulsifying perfluorotertiary amine. It was found, however, that the cells were actually growing on a layer of protein desorbed from the serum used in the nutrient medium. This layer was not stable and growth was erratic. Moreover, the cultures had to be vigorously stirred to ensure equilibration with oxygen resulting in cellular damage. Finally, since serum contains very little extracellular matrix material, the microspheres did not provide a good substratum for anchorage-dependent cell growth. In fact, growth on these microspheres was not as good as on commercially available microcarriers fabricated with collagen, or gelatin.

A recent method of cultivation of anchorage-dependent cells teaches growing the cells on a perfluorocarbon reservoir bonded to a perfluoroalkylated cell binding protein. The PFC substratum delivers oxygen directly to cells at the cell-substratum interface, a region where oxygen is severely limited when cells are grown in polystyrene cell tissue culture dishes. The system consists of perfluoroalkylating an attachment protein such as gelatin and bonding it to a PFC such perfluorodecalin (PFD). Hela cells and HEP G2 cells on these substrata were found to grow beyond the monolayer stage forming more than 19 layers of cells. The number of layers formed depended on the amount of oxygen available from the PFC. The results showed that multilayer growth depends on a system where oxygen is delivered to both the substratum and the medium.

However this method of cell culture also has significant drawbacks. For example, it teaches the use of PF-alkylating agents such as PF-octyl isothiocyanate which require the use of organic solvents. Many organic solvents are toxic both to cells in culture and the persons who use them. Additionally, cell binding proteins such as matrix factors have a critical conformation which may be altered by organic solvents thereby reducing the ability of cells to bind to the perfluorinated proteins.

The previously used perfluoro-alkylating agents do not efficiently couple proteins to PFCs. First, the agents are unstable and must be prepared fresh for each time they are used. The agents also poorly bond matrix factors to PFCs, perfluorinating only 24% of the amino groups. Finally, the previously taught PF-alkylating reaction is very slow and may take several days to coat one substratum.

The dishes or flasks used to grow cells in culture are another factor limiting the amount of oxygen available to cells. It has long been recognized that cells growing in conventional tissue culture dishes or flasks are not provided with the level of oxygen they need to maintain optimal growth and function. The cell culture dishes are typically made of plastic or other materials which are impermeable to air. Thus, once the cells are seeded in the culture dishes only a finite amount of oxygen is available and cannot be replenished. Because of the lack of oxygen, cell growth is typically limited to a monolayer when cultured in plastic dishes or flasks.

Some attempts have been made to replenish the oxygen in a culture dish. However, cell culture dishes have not been developed which continuously and selectively supply the oxygen to the growing cells without stirring, bubbling or otherwise disrupting the cells. Moreover, some of the plates and techniques for replenishing oxygen require media to be withdrawn and replenished which creates a potential for contamination of the culture. Because of the lack of oxygen, the cells dedifferentiate and lose of much of their characteristic biochemistry and morphology. This limits the amount of information which can be gained from investigations in cell biology or clinical science since many processes which depend on good rates of oxygen uptake are depressed or lost. The usefulness of tissue cultures for studying problems involved in basic cell science or clinical medicine has been seriously limited by the fact that cells from established cell lines have very little similarity to the cells in the organs from which they were derived.

From the foregoing, it will be appreciated that it would be an advancement in the art to provide a system which provides improved oxygenation to anchorage-dependent tissue culture cells. It would be a further advancement in the art if the anchorage-dependent cells were able to form multilayer tissue-like structures. It would be yet another advancement in the art if the improved oxygenation could be accomplished without mechanically stirring or agitating the culture media It would also be an advancement in the art if the increased oxygenation resulted from perfluorocarbon molecules that were not in direct contact with the tissue culture cells. It would be an advancement in the art if the number of free radicals produced in the culture media were greatly reduced or eliminated. It would be another advancement in the art to provide a method for perfluoroalkylating proteins that would not alter the critical conformation of the proteins. It would be an additional advancement in the art if the method for perfluoroalkylating proteins did not use organic solvents. It would be a further advancement if the method for perfluoroalkylating proteins could be carried out in an aqueous solution. It would also be an advancement to provide a rapid method for perfluoroalkylting proteins. It would be a further advancement in the art to provide a stable agent for perfluoroalkylating proteins. It would be a further advancement in the art if the agent for perfluoroalkylting proteins could efficiently perfluorinate the amino groups of proteins. It would be another significant advancement in the art to provide a cell culture dish that would allow for the continual replenishment of oxygen. It would be a further advancement if the oxygen could be replenished without disturbing the cells growing within the dish.

Such methods and systems are disclosed herein.

### 3. BRIEF SUMMARY OF THE INVENTION

The present invention is directed to novel oxygenation systems which support growth of anchorage-dependant cells. More particularly, the invention relates to a substratum for growing culture cells in vitro which is capable of forming three-dimensional tissue-like structures. A surface such as a cell culture dish is covered with a reservoir of perfluorocarbon (PFC). A volume of perfluro-aldehyde (PF-aldehyde) is then mixed with the PFC. The PF-aldehyde automatically reorients in the solution so that the aldehyde head groups are at the organic-aqueous interface and PFC-tails of the PF-aldehyde bind in the PFC reservoir. The PF-aldehyde is anchored in the PFC and a strong, stable surface is created for cell growth. A matrix protein or other protein to which cells can attach is bound to the aldehyde head of the PF-aldehyde creating a surface on which anchorage-dependant cells attach and grow. An aqueous growth media may be added over the PF-aldehyde/protein interface and anchorage-dependent cells are seeded in the medium and allowed to grow.

Perfluorocarbons are organic compounds in which all hydrogen atoms are replaced by fluorine atoms. The carbon-fluorine bond of the PFCs is so strong that they are very stable and inert for biological purposes and do not produce free radicles. Oxygen is 15 to 20 times more soluble in PFCs than in water, making PFCs useful to oxygenate cells in culture. Examples of PFCs that maybe used in the current invention include, but are not limited to, perfluorotrihexylamine (FC-71), perfluorodecalin, perfluorortibutylamine, perfluorotripentylamine (FC-70), and other high molecular weight perfluoroamines. Because the strength of the PFC reservoir may affect cells' ability to produce three-dimensional structures, amore viscous PFC such as FC-71 maybe used in certain embodiments. In other embodiments, the PF-aldehyde may be desorbed on surfaces of polytetrafluoroethylene (Teflon) or introduced into the melt process of the Teflon production. Such a configuration will provide a stiff surface on which the cells may grow.

In one embodiment of the invention a PF-aldehyde is coupled to an attachment factor such as gelatin, collagen, albumin, fibronectin, or poly-1-lysine. The PF-aldehyde may be coupled with the free amino groups on proteins by using cyanoborohydride via Schiff reaction. The attachment factors are coupled to the PF-aldehyde The PFC-tails of the PF-aldehyde anchor the attachment factor in the PFC reservoir.

The length of the PFC tail of the PF-aldehyde determines the strength of the bond between the PFC reservoir and the PF-aldehyde. The longer the PFC tail the stronger the bond. Therefore, the PF-aldehyde having at least eight terminal perfluorinated carbons may provide a sufficiently strong bond. A perfluoro-aldehyde with a number of terminal perfluoxinated carbons in from about eight to about thirty may provide a sufficiently strong bond. For example, in one embodiment of the invention a 17-PF-aldehyde has been synthesized and used.

Complex substrata with rarer matrix factors are required for optimal growth of many types of differentiated cells. Amore complex substrata maybe prepared if the PFC is first covered with poly-1-lysine. The poly-1-lysine has many free amino groups which maybe coupled to some of the rarer matrix factors using glutaraldehyde. These rare matrix factors include but are not limited to laminin, entactin, and proteoglycans, or mixtures of these factors. To prepare an environment for the growth of the cells similar to that found in the body, other factors can be coupled to the PF-aldehyde with an appropriate coupling agent at their optimal pH.

With use of the present system, eukaryotic cells maybe grown to form multiple cell layers. Many anchorage-dependent cells such as Hep G2 cells, hepatocytes, liver cells, kidney cells, brain cells, spleen cells, stem cells, cancer cells, bone marrow cells, nerve cells, and heart cells may benefit from the increased oxygenation and produce tissue-like structures. Additionally, cells may be co-cultured; that is a cell or tissue under study may be simultaneously cultured with other cell types from the same or a different organ which produce many materials which the cell or tissue under study may need. These materials are not otherwise available to add to the culture.

To support the increased metabolism of the multilayered culture, the aqueous growth medium maybe supplemented. In one embodiment, the cells are given a supplemented growth medium. The basic medium consists of a revised Dulbecco's modified Eagles's medium supplemented with 10% fetal calf serum, insulin, EGF, transferrin, and pyruvate (DMEM+). To the DMEM+is added insulin, transferrin, progesterone, corticosterone, triiodthyronine, vasopressin, galactose, 2-phosphoascorbate, phosphocthanolamine, putrescine, Vitamin B 12, biotin, Vitamin E, calcitriol, ergocalciferol, ergothioneine, acetyl carnitine, acetyl cysteine, selenium, ZnSO₄.7H₂O, CuSO₄.5H₂O, MnSO₄, progesterone, and testosterone.

The present invention also relates to a novel culture plate which allows for the controlled and continuous oxygenation needed for the optimal growth of anchorage-dependent cells. The plate has been named the Controlled Oxygenation Perfluorocarbon System plate or COPS plate. The COPS plate has a shell which encloses a chamber. An inlet port and an outlet port may be provided within the walls of the shell, allowing for selectable gas exchange within the chamber. At least one well for cell growth is provided within the chamber. A protein-covered PFC substratum is layered with an aqueous growth media, and cells are seeded within the well.

The well has a well wall and a bottom. The well may be made of an oxygen permeable material so that oxygen from the chamber may diffuse through the wall into the PFC contained in the well. In one embodiment the well wall is made of silicone, which is permeable to oxygen. The well bottom can be constructed of an optically clear material so that the cells maybe observed with an inverted microscope. The well wall maybe made from a section of silicone tubing cut into an appropriate length. The silicone tubing may be platinum or peroxide treated.

A constant level of oxygen may be provided to the cell culture by incubating the plate within an incubator with a fixed level of oxygen with the inlet and outlet ports left open. In other embodiments, the inlet and outlet ports maybe attached to a ventilation system to maintain a desired oxygen level within the chamber.

This novel tissue culture technique and culture plate overcomes several problems facing the art. First, the PF-aldehyde protein/PFC substratum of the present invention is capable of continually supplying high concentrations of oxygen to the cultured cells. The high affinity of PFC for oxygen permits high concentrations of oxygen to pass through the PF-aldehyde/protein interface and reach the culture cells. Moreover, the COPS plate allows PFC to be continually oxygenated without disturbing the growing cells and without contaminating the cells. No mechanical stirring or agitation is required. Depending on the intended use, the wells with a PFC reservoir can either be a closed system which is supplied with a finite concentration of oxygen or an open system which is continuously regenerated with oxygen at ambient concentrations.

In one embodiment, collagen was perfluorinated by reacting a PF-aldehyde in a FC-71 substratum with the free amino groups on the collagen. Hep G2 cells were seeded on the collagen coated FC-71. The Hep G2 cells grew to a density of more than 10⁷ cells/cm² forming multilayered structures. Moreover, the Hep G2 cells continued to secrete albumin, a marker for differentiation, at much higher levels than obtained in standard polystyrene dish cultures.

Also, the system of the present invention limits free radicals. As discussed above, free radicals are toxic to mammalian cells at even very low concentrations. PFCs do not contain free radicals. Moreover, the PFC which delivers oxygen to the cells never comes in direct contact with the growth media. The PFC and the aqueous growth media are separated by the PF-aldehyde-matrix coat layer. Thus, the oxygen is not as available for reaction with the iron, copper, and other metabolites in the growth media which produce free radicals. Additionally, the medium can be supplemented with a high concentration of antioxidants.

Furthermore, the PFC is never in direct contact with cells. PFCs have been shown to adversely affect cells in culture. In the system of the present invention, the PFC reservoir is separated from the cells by the PF-aldehyde-protein interface on which the cells grow eliminating harmful effects of PFCs on the cells.

Because the system uses a PF-aldehyde to perfluorinate the matrix proteins, no organic solvents are required, and the conformation of the proteins is not altered. The PF-aldehyde also allows the rapid coupling of proteins which may be performed in aqueous solution. The PF-aldehyde is very stable and can be stored for months in a freezer without change.

The foregoing elements of the cell culture system have resulted in a novel system for growing cells which yields three dimensional tissue-like structures never before achieved in culture. These and other advantages of the present invention will become apparent by examination of the following description of the accompanying drawings, the detailed description of the invention, and the appended claims.

### 4. BRIEF SUMMARY OF THE DRAWINGS

Amore particular description of the invention briefly described above will be rendered by reference to the appended drawings and graphs. These drawings and graphs only provide information concerning typical embodiments of the invention and are not therefore to be considered limiting of its scope.

FIG. 1 is a perspective view of one embodiment of a COPS plate of the present invention.

FIG. 2 is an exploded view of a well of the COPS plate of FIG. 1.

FIG. 3 is across sectional view of one embodiment of the present invention illustrating cells grown on a PF-aldehyde/protein substratum in a well of the COPS plate of the present invention.

FIG. 4 is a graph illustrating albumin secretion by Hep G2 cells grown in COPS plates on different substrata. Black squares represent secretion by cells grown on collagen-coated PF-71. Black circles represent secretion by cells grown on poly-1-lysine-coated PF-71. “DIV” refers to “days in vitro.”

FIG. 5 is a graph illustrating albumin production in Hep G2 cells. Black squares represent production in COPS plates with improved nutrient media Black triangles represent production in polystyrene tissue culture dishes with improved nutrient media Black circles represent production in polystyrene tissue culture dishes with standard media. “DIV” refers to “days in vitro.”

FIG. 6 is a graph illustrating albumin production in Hep G2 cells grown in COPS plates with different media. Black circles represent production with improved nutrient media. Black diamonds represent production with standard media. “DIV” refers to “days in vitro.”

FIG. 7 is a graph illustrating Hep G2 growth (cells/cm²) on different substratums. Black circles represent growth in COPS plates of the present invention. Black squares represent growth in polystyrene plates. “DIV” refers to “days in vitro.”

### 5. DETAILED DESCRIPTION OF THE INVENTION

The present invention is directed to novel oxygenation and cell culture systems which support the growth and differentiation of anchorage-dependent cells. A novel perfluorocarbon (PFC) containing an aldehyde head group has been synthesized. This perfluoro-aldehyde provides a rapid and efficient agent for bonding adhesion factors and other proteins to PFC's. The adhesion factor promotes good adhesion and growth, while the PFC provides oxygen directly to the cells at the cell-substratum interface, a region that is severely hypoxic when cells are grown on conventional tissue culture plates. The lack of oxygen limits their growth to a monolayer and restricts expression of many cell-specific functions. The PF-aldehyde makes it possible to fabricate substrata on which cells can grow on an optimally coated surface while being provided with optimal levels of oxygen. Cells cultured on these matrix-coated PFC substrata have been found to grow beyond the monolayer stage to form multilayer tissue-like structures with cells expressing increased levels of cell-specific proteins.

The PF-aldehyde perfluorinates free amino groups on proteins via the Schiff reaction with cyanoborohydride. This reaction provides perflourinated carbon chains which sink into the PFC, binding the proteins firmly to a surface such as a tissue culture plate. Since the strength of the bonding would be expected to increase with chain length, the PF-aldehyde may have a carbon chain length in the range from about eleven to about thirty-three. A carbon chain length in the range from about 11 to about 17 can provide sufficient binding strength. For example, in one embodiment a carbon chain length of about 17 carbons has been used. The number of perfluorinated carbons within the chain may vary, however, PF-aldehydes having a number of perflourinated carbons of at least about eight have been successfully used. A PF-aldehyde with a number of terminal perfluorinated carbons in the range from about eight to about thirty can be used. For example, a PF-aldehyde with about 17 perfluorinated terminal carbons has been successfully employed.

To synthesize the PF-aldehyde, a perflourinated alcohol is dissolved in a mixture of methylene chloride and trifluorotoluene and incubated in the presence of the Des-Martin reagent at room temperature. This coverts the alcohol to the aldehyde. The reaction maybe represented as follows where RX_{fX} represents a perfluorocarbon group of X carbons with the terminal X carbons perflourinated:

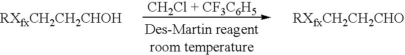

The reaction is terminated with the development of color and is maximal in one hour. Diethyl ether is added to precipitate all reagents except the perfluoro-aldehyde. This is washed with saturated NaHCO₃ with a seven-fold excess of sodium thiosulfate and then dried over MgSO₄. The PF-aldehyde is very stable with a long shelf life. Preparations that have been stored more than a year at −20° C. show no decrease in activity.

This procedure can be easily modified so as to provide perfluoro-aldehydes with a length of up to about twenty perfluorinated carbons. The method used above or a two phase reduction of long chain alcohols dissolved in PFC may be used to produce PF-aldehydes. The increased length of the perfluorinated carbon tails would anchor the proteins even more firmly to the surface. This could be advantageous for growing cells that may exert high traction forces on the surfaces during growth.

The PF-aldehyde is mixed by a 20 minute treatment with ultrasound with the PFC to be used as the oxygen reservoir. This results in the perfluorinated carbon tails being solubilized in the PFC and the hydrophilic aldehyde head groups arrayed at the interface. This can be done in batch quantities and used two to three weeks after the treatment with ultrasound. A small volume, 0.7 ml, of this mixture is added to the well of a culture plate. The aldehyde head groups automatically reorient to the surface after the pipetting. The surface is covered with 0.7 ml of a solution containing an adhesive factor dissolved in 0.4M sodium borate containing cyanoborohydride at three times the molar concentration of aldehyde groups. Adhesive factors may include but are not limited to proteins such as gelatin, albumin, collagen type 1 and type 4, fibronectin, as well as poly-1-lysines. More complex substrata can be prepared if the PFC is first covered with poly-1-lysine. This leaves any ammo groups free for coupling with some of the rarer matrix factors, such as laminin, entactin, and proteoglycans, or mixtures of these factors with collagen, using glutaraldehyde or cyanobrohydride at appropriate pH or other coupling agents. Complex substrata are required for optimal growth of many types of differentiated cells. The PF-aldehyde provides a simple and versatile method for preparing such complex substrata. This would be faster and cheaper than procedures now used to isolate Matrigel and other natural intercellular adhesive complexes from tissues.

The pH of the coupling mixture depends on the solubility characteristics of the matrix factor. For example a pH of about 4.0 can be used when bonding collagen, and a pH of about 7.5 can be used when bonding poly-1-lysines. The reaction is allowed to proceed at room temperature for 1-2 hours. The coupling solution is aspirated off and excess reagents removed by washing with water. The substrata are covered with a serum-supplemented medium and annealed overnight or longer, if convenient, by incubating at 37 C. This method has provided substrata supporting multi layer growth of Hep G2 cells, Hela cells, Vero cells and primary rat hepatocytes.

Cells cultured using the method of the present invention are able to grow more like cells in vivo as compare to standard plastic tissue culture plates. Because of the coupling technique, the cells adhere to matrix factors. The matrix factors occupy less of the cells' surface than when the cells are bonded to aplastic culture dish. The adhesion of the cells to the matrix factors is weaker and more specific that the nonspecific adhesion on plastic. This weaker specific adhesion allows the cells to round-up and interact with each other. Additionally, the cells will continue to grow on top of each other so long as sufficient nutrients and oxygen is supplied. The PFC may provide the cells with a degree of polarity, needed for normal cell function, similar to basement membranes in vivo.

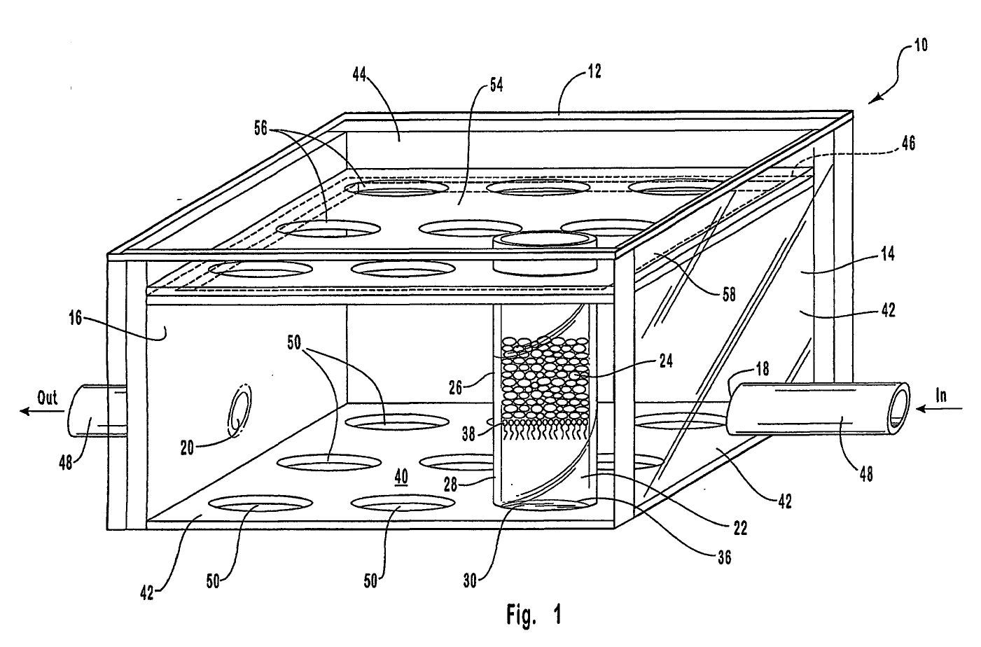

Referring to FIG. 1, a system 10 for growing anchorage-dependent cells is presented. The system 10 has a multi-well controlled oxygenation perfluorocarbon system plate 12 (COPS plate) which may be used in conjunction with the PFC substrata 22 for cell culture. In ceratin embodiments, the COPS plate 12 has a shell 14 made of Lexan or another material which can be sterilized by autoclaving. The shell 14 encloses a chamber 16 with an inlet port 18 and an outlet 20 port for gas exchange. Any desired level of oxygen within the chamber 16 may be maintained by attaching the inlet port 18 and outlet port 20 to a ventilation system (not shown). The inlet port 18 and outlet port 20 may also be left open in an incubator (not shown). For example if the ports 18, 20 are open in a 5% CO₂-air incubator, cells 24 are provided with a constant level of 20-21% oxygen. If it is desired to grow cells 24 within a closed system, the ports 18, 20 maybe closed thereby allowing only the oxygen contained within the chamber 16 to be used by the cells.

The COPS plate 12 has one or more wells 26 for cell growth. The wells 26 have a well wall 28 and a bottom 30. The well walls 28 are constructed of a material which is both permeable to oxygen and capable of holding the PFC 22 and aqueous growth media 32. For example, the wells 28 maybe constructed of silicone. Cells 24 growing in the wells 26 are provided with oxygen by diffusion from the chamber 16 through the well wall 28 and into both the PFC 22 and the aqueous growth medium 32. Because PFC's 22 are optically clear, the well bottom 30 maybe constructed out of a material such as optically clear glass to allow for observation of the cells 24 using a standard inverted microscope.

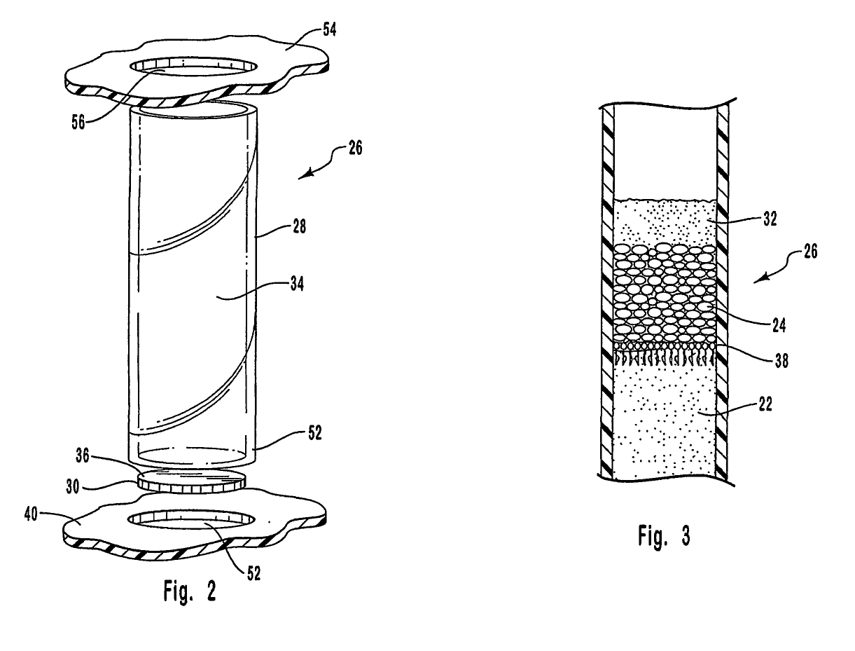

Referring now to FIGS. 2 and 3, the wells 26 can be made with silicone tubes 34 which have been fitted with a bottom 30 of optically clear glass discs 36. Silicon is both permeable to oxygen and capable of holding both the PFC 22 and growth media 32. Oxygen is delivered to the cells 32 both at the cell substratum interface 38 from the PFC 22 and from the aqueous medium 32, and it is replenished in both the PFC 22 and the medium 32 as it is taken up by the cells 14. Since oxygen is 15-20 times more soluble in PFC's than in aqueous solutions such as growth medium, the PFC 22 provides a reservoir, or head of oxygen which stabilizes the pO₂ during growth up to very high densities.

A small volume of a protein 38 covered PFC substratum 22 may be placed in the well 26. Nutrient growth medium 32 maybe layered over the PFC substratum 22, and a suspension of cells 24 seeded in the well 26.

The COPS plate 12 maybe constructed by assembling a bottom plate 40 with four side walls 42. The plate may sized and proportioned according to the desired use. For example in one embodiment, the bottom plate 40 is 3 inches square and the side walls 42 are 3 inches long and 1 1/2 inches high. The sidewalls 42 and bottom plate 40 may be made of Lexan or another material which can be sterilized by autoclaving. The assembled plate 12 is then autoclaved to sterilize.

The COPS plate 12 is fitted with a lid 44. In the illustrated embodiment, the lid 44 is slightly larger than the COPS plate 12 to allow for strips 46 to be fastened at the edge of the lid 44. For example, in one embodiment, the COPS plate 12 is 3 inches square and the lid 44 is 3½ inches square. Strips 46 which are about are connected to the outer edges of the lid 44 so that it fits over the body of the plate 12 preventing contamination of the chamber 16 and wells 26 and escape of gasses.

The plate 12 is provided with holes 18,20 drilled in opposite walls of the plate for an inlet port 18 and an outlet port 20 to allow for the exchange of gasses. The inlet port 18 and the outlet port 20 can be fitted, if desirable, with microvalves 48, for flushing the plate 12 with special gas mixtures.

In one embodiment, the COPS plate 12 has nine wells 26. The wells 26 are spaced evenly within the chamber 16. However, the COPS plate 12 may have any number of wells 26 in a variety of configurations without departing from the scope of the present invention. The wells 26 maybe constructed by first drilling holes 50 into the bottom plate 40.

The holes 50 create a base for the well 26 which may be constructed as follows. Silicone tubing 34 is washed with 1% alconox. An optically clear glass disc 1.32 cm±0.024 mm in diameter and about 1.66 mm±0.025 mm thick (Precision Scientific Co. San Francisco, Calif.), is inserted perpendicularly into the tube 34 to a depth such that, with a slight push sideways, one edge of the disc 36 is flush with the inside bottom rim 52 of the tube 34. The entire disc 36 is then pushed flat by gentle pressure from inside the tube 34 with a glass rod (not shown). The glass disc 36 together with the bottom rim 52 of the silicone tube 34 is then pushed by thumb upward into the hole 50, so that both are flush with the bottom 40 of the plate 12. If it is necessary to adjust the position of the disc 36 so that it is flat and flush with the bottom 40, the disk 36 may be tapped from the inside of the well 26 with a glass rod. The procedure takes advantage of the fact that silicone tubing 34 can be both stretched to insert the glass disc 36 into the tube 34 and compressed to fit the tube 34 with disc 36 inside the hole 50. Assembling nine wells 26 takes about five minutes. The method provides leak-proof wells 26.

Silicone tubes 34 are not perfectly straight, which may interfere with observation of the growing cells 24. Therefore, in certain embodiments a second Lexan plate 54 with nine holes 56 is placed over the tubes 34 and rested on struts 58 which have been attached on opposite side walls 42 of the shell 14.

After the multiwell plate 12 has been assembled, it is autoclaved and then dried in an oven at 56° C. The glass bottoms 30 of the wells 26 may then be covered with a small volume, about 0.7 ml, of protein-covered PFC substratum 22, the substratum covered with a nutrient growth medium 32, and the medium 32 seeded with a suspension of cells 24.

The particular dimensions of the plate 12, walls 42, and struts 58, which have been given, can be changed to accommodate a different number of wells 26. However, the relative size of the silicone tubes 34, holes 50, and glass discs 36 should be maintained in order to obtain leak-proof and mechanically stable wells 26.

As cells are provided additional oxygen, the cells grow to form multilayer structures. The number of layers of cells which can be grown depends directly on the amount of oxygen provided. This indicates that multilayer growth is the result of increased metabolic competence of cells due to the increase in respiration. However, with the increased metabolic competence comes a requirement for increased nutrients. More layers of cells maybe grown by using a growth medium which provides the nutrients and anti-oxidants needed for the increased metabolic demands of cells with higher rates of respiration. A novel nutrient growth medium has been developed which supports growth of more than twenty layers of anchorage-dependent cells like Hep G2 cells. The nutrient medium also supports growth of several layers of primary rat hepatocytes. Moreover, the cells continue to secrete albumin, which is a marker for maintenance of differentiation.

Attachment-dependent cells such as Hep G2 cells grown in conventional tissue culture plates stop growing once a monolayer has formed. Moreover, the respiration-dependent synthetic mechanisms are suppressed because the cells are subjected to hypoxia.

When cells are cultured on the protein-covered PFC substratum of the present invention, they continue to grow after the monolayer stage to from multilayers of cells secreting high levels of albumin. The PFC substratum provides higher and more uniform levels of oxygen than can be provided in polystyrene tissue culture dishes. This results in an increased rate of respiration and increases in respiration dependent synthetic mechanisms which would be suppressed in cultures using plastic dishes where cells are subjected to varying degrees of hypoxia. It has now been found that full expression of these mechanisms, and optimal 3-D growth of Hep G2 cells, depends not only on providing the high levels of oxygen needed but also on providing a nutrient medium which can meet the demands of an activated aerobic metabolism.

A screening study was done to determine factors which might be required for optimal multilayer growth. The basic medium consisted of a revised Dulbecco's modified Eagles's medium supplemented with 10% fetal calf serum, insulin, EGF, transferrin, and pyruvate (DMEM+). The three hormones have been shown to promote growth of hepatocytes in serum free medium. DMEM+ does not contain Dexamethasone (Dex), which is universally used in primary cultures of rat hepatocytes, in which Dex is found to briefly stimulate synthesis of DNA. Dex has an exceptionally high affinity for the super steroid receptor family which would prevent normal interactions of the physiological steroids important for long term growth and multilayer formation. Tests in which DMEM+was supplemented with various factors, and mixture of factors, revealed fifteen supplements which increased the number of layers of hep G2 grown on PFC substrata using the COPS plate. These studies resulted in the development of a novel medium which supports more than twenty layers of albumin secreting Hep G2 cells and several layers of primary rat hepatocytes.

The medium contains a mixture of progesterone, testosterone, estradiol and corticosterone; a mixture containing Tri-iodothyronine, Vitamin D3 and/or very high concentrations of ergocalciferol, and metabolites, such as acetylcarnitine and pyruvate; and a mixture of seven naturally occurring anti-oxidants. These supplement aid in maintaining the CytP540 system and general health of the endoplasmic reticulum, regulating activity and transport mechanisms of the mitochondria, and protecting cells against injury due to free radicals which would increase with the increase in respiration. The serum supplement provides additional factors, such as carriers for hormones, lipids, and micro nutrients which, present even at only 10% of the level available to cells in vivo, are essential. Cells grown in serum-free medium do not produce multilayers.

The medium does not significantly improve growth of cells in monolayer culture. This indicates that growth of multilayers depends on oxidative mechanisms which are not involved in monolayer growth. It also indicates that growth is limited to a monolayer because of poor oxygenation and failure to meet the metabolic demands of cells with higher rates of respiration.

All publications, patents, and patent applications cited herein are hereby incorporated by reference.

### EXAMPLES

The following examples are given to illustrate various embodiments which have been made with the present invention. It is to be understood that the following examples are not comprehensive or exhaustive of the many types of embodiments which can be prepared in accordance with the present invention.

### Example 1

Cell Culture

Hep G2 cells were obtained from the American Type Culture Collection (ATCC). The Hep G2 cells were carried as stock cultures in DMEM supplemented with 1 mmol sodium pyruvate, 0.1 mmol non-essential amino acids, 10% fetal calf serum, 50 μg/ml streptomycin, and 50 μg/ml penicillin. FBS was obtained from Hyclone Laboratories, Logan, Utah. All the biochemicals were obtained from Sigma. The cells were passaged using 0.25% trypsin and EDTA and split every 5 to 7 days at a ratio of 1:4. Trypsinized suspensions of the stock cultures usually contained clumps of cells and were not suitable for quantitative growth studies. The suspensions used in the growth experiments were obtained by trypsinization of one of the sub-cultures two to three days after passage. These provided single cell suspensions with a viability of more the 99% as determined by exclusion of trypan blue.

### Example 2

Chemicals and Plastics

Perfluorotrihexylamine (FC-71) was obtained from 3M. The 3-Perfluorooctylpropanol was obtained from Fluorochem. Cyanoborohydride other chemicals used in synthesis of the PF-aldehyde were obtained from Aldrich Chemicals. Silicone tubing was obtained from Norton Chemicals. Lexan plastic and micro-solder iron (MCB Electronics, Centerville, Ohio) were obtained from a local plastic supply store. The machining of the Lexan was done in a standard machine shop.

Plastics may be soldered using a standard plastic a soldering iron obtained, e.g., from MCM Electronics.

### Example 3

Culture Analysis

Cell growth was determined by fluorimetric assay of DNA using the Hoechst stain 33285 (Labarca, 1980) and calf thymus DNA as standard. This was correlated with cell number by comparison with a calibration curve for cell number versus DNA made from many trypsinized suspensions.

Albumin secretion was assayed by sandwich ELISA using a monoclonal anti-human albumin antibody and a horse radish peroxidase-conjugated polyclonal goat anti-human antibody (Bethyl Labs-A 80-129P) and TMB peroxidase stain (Kirkegaard and Perry Labs no. 50-76-00). The development of color at 650 nm was maximal after 20 minutes, at which time the reaction was stopped by addition of phosphoric acid to give a final concentration of 0.1M. The yellow color developed was read at 450 nm using a Bausch and Lomb Spectronic 20. The amount of albumin secreted was determined by comparison with a calibration curve made using human albumin.

### Example 4

Synthesis of Perfluoroaldehyde (PF-Aldehyde)

A PF-aldehyde was synthesized by converting a PF-alcohol to a PF-aldehyde. The overall method of synthesis involves converting 3-(Perfluorooctyl)propanol to an aldehyde using the Des-Martin oxidizing agent. This reagent was prepared according to the revised procedure of Ireland and Liu (1993). 2-iodobenzoic acid is oxidized by KBrO₃ to the hydroxyiodinane oxide (compound 2) and then treated with acetic anhydride and 0.5% TsOH (p-toluene sulfonic acid) at 80° C. This is complete in about 2 hours with a yield of 90% of the reagent recovered as a precipitate. The agent should be prepared without interruption since it has been reported that compound 2 may be explosive if stored. The aldehyde was then prepared by adding 0.67 g of the Des-Martin reagent dissolved in 20 ml of dried methylene chloride to 2 g of the 3-(Perfluorooctyl) propanol. The mixture is stirred for one hour at room temperature in an atmosphere of nitrogen. 20 ml of ether is added, and the mixture is washed with 75 ml of water saturated with NaHCO₃ and a seven fold excess of sodium thiosulfate is added. The mixture is stirred until two clear layers separate. The aqueous layer is removed, and the organic layer is washed with another 75 ml of water saturated with sodium bicarbonate. The aqueous layers are combined and any residual product is extracted with ether. The combined organic phases are dried with anhydrous magnesium sulfate, then filtered through a glass frit to remove the drying agent and evaporated to a syrup under reduced pressure. The PF-aldehyde is purified by distillation using a water jacketed short path distillation head (Fisher Scientific, no A9199947945) while heating the bulb with a heat gun (90° C.). The yield of the aldehyde, P_{F8}CH₂CH₂CHO, is from 70-80%. Another longer PF-aldehyde P_{F17}CH₂CH₂CHO has been synthesized in the same manner with similar yields. These aldehydes are a very efficient PF-alkylating agent. Assays found that 86-90% of the free amino groups on gelatin were PF-alkylated compared to 14% PF-alkylated when perfluorooctylpropyl isocyanate was used. The aldehyde is very stable and can be kept for more than six months at −20° F. without change as determined by NMR spectroscopy.

### Example 5

Preparation of FC-71 Used in Fabrication of Substrata

The PF-aldehyde is miscible, using ultrasound, in PFCs resulting in a solution in which the perfluorinated carbon tails are firmly anchored in the PFC and the aldehyde head groups are arrayed at the interface. These can then be coupled via the free amino groups on matrix factors or other proteins, using cyanoborohydride. It was found that the concentration of both the PF-aldehyde added to the PFC and the concentration of the matrix factor used in the coupling step was important and different for the different factors which have been tested. It was also found that with all factors tested, better substrata were formed when the PFC had first been pre-treated for 30 minutes with ultrasound (Branson 200 ultrasonic cleaner, 120 V, 50/60 Hz, 40 W). The mechanisms involved are not understood, but one ostensible effect is that it releases gases from micro bubbles in the PFC's, preventing the formation of bubbles forming later in the substrata after the cells have been seeded. It may also disperse conglomerates of some of the PFC isomers, allowing amore even distribution of the PF-aldehydes. The preliminary treatment with ultrasound can be done on a large volume of FC-71 and does not have to be repeated. The pre-treated FC-71 is sterilized by autoclaving for 20 minutes.

### Example 6

Preparation of Collagen Coated FC-71 Substrata

A stock solution of PF-aldehyde at 30 mg/cc is dissolved in the FC-71. A volume, 0.055 ml, of this solution is added to 6.45 ml of the pre-treated FC-71 contained in a 30 ml Nalgene bottle. The mixture is treated with ultrasound for twenty minutes using the Bronson 200 model cleaner. A volume of 0.7 ml of the mixture is carefully pipetted into each of the wells in a COPS plate 5 which has been sterilized by autoclaving and dried. The mixture automatically re-orients so that the aldehyde head groups are at the interface.

A volume of collagen type 1 is added to a solution of cyanoborohydride at 1.4 mg/ml in 0.4M sodium borate buffer (pH 4.0) to give a concentration of 50 μg of collagen per well. A volume of 0.7 ml of this solution is added to each well on top of the 0.7 ml of the PF-aldehyde/FC-71 mixture. The plate is incubated at 37° C. for 2-3 hours.

The cyanoborohydride-collagen solution is aspirated out of the wells. Care should be taken to leave a small volume so as not to disturb the substrata. Residual collagen, borate and cyanoborohydride are removed by rinsing three times with about 1 to 1.5 ml of 0.01 M acetic acid followed by two rinses with water. It maybe convenient to add a pH indicator such as phenol red to the rinses so as to be able to see the substratum interface. The substrata are covered with 1 ml of a nutrient containing 10% serum and 2.2 g/L of NaHCO3. The plates are incubated overnight in a 5% CO₂ air incubator at 37° C. to anneal the substrata and equilibrate with gases. The medium is aspirated off and the plates are ready to be seeded with cells. If not used immediately, the plates can be left covered with the serum-supplemented medium for as long as three weeks before being used.

### Example 7

Albumin Secretion by Hep G2 Cells

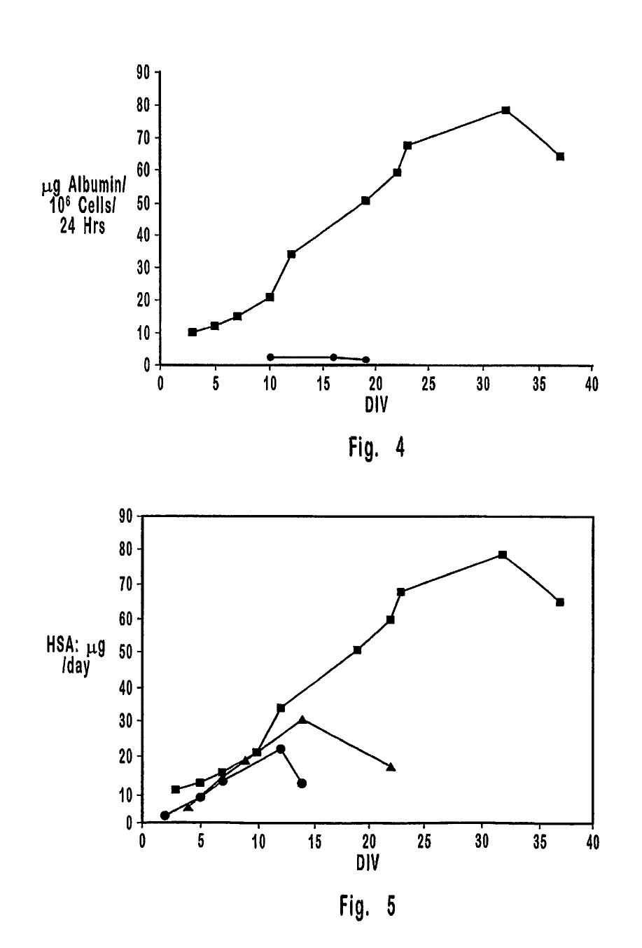

Culturing Hep G2 cells on collagen-coated or poly-1-lysine-coated PF-71 substrata results in significant differences in the amount of albumin cells excrete during growth and the density of cells that can be reached. FIG. 4 presents data obtained from five suspensions grown in COPS plates. The wells were seeded with 30,000 cells/well on substrata coated with collagen, as in previous experiments, or with high molecular weight poly-1-lysine. Preliminary experiments had shown the optimal conditions for coupling, with respect to the amount of PF-aldehyde in the PFC base, the concentration of poly-1-lysine and pH. Both sets of plates were incubated open in a 5% CO₂-air incubator. Two wells from each set were sampled at the times indicated and assayed in duplicate for albumin and DNA. The variations between duplicate assays was less than 10%. The albumin secreted by cells growing on the collagen coated-substrata was found to increase with time, reaching 34-76 μg/106 cells/day after 12 and 15 days respectively. In contrast, albumin secretion from cultures grown on poly-1-lysine substrata was negligible reaching a maximum value of only 3 μg/10⁶ cells/day after 19 days. Assays for DNA showed that this was not correlated with decrease in growth, which was found to be somewhat greater in cultures on poly-1-lysine than in the collagen substrata. On the poly-1-lysine substrata, the cell density was found to be 7.2×10⁶ cells/cm² and at 16 days and 8.3×10⁶/cm² at 19 days compared to a density of 3.3×10⁶ cells/cm² at 23 days and 6.4×10⁶/cm² after 35 days on collagen substrata. The cultures on poly-1-lysine have not been carried beyond 19 days.

The significant difference in albumin secretion on the two substrata cannot be attributed to differences in the amount of oxygen available. Both sets of cultures were grown with a 20-21% level of oxygen that was sufficient to support growth to high densities on both substrata. This strongly suggests that the substrata affect the way the energy available to the cells is metabolically allocated. Poly-1-lysine and collagen substrata may differentially affect the expression of glucose transporters. Regardless of the mechanisms involved, in view of these new findings, the two substrata may significantly affect the expression of cell surface-specific antigens, which could be of major importance for vaccine production.

### Example 8

Nutrient Medium for Supporting Multilayer Growth and Cell Differentiation

DMEM+ has been found to support indefinite propagation of Hep G2 cells as monolayer cultures. This indicates that all of the factors required for proliferation of cells at low densities have been met. Supporting optimal growth of cells in better oxygenated cultures could depend on additional factors involved in respiration-dependant mechanisms. In addition, many essential factors provided at only 10% of those present in plasma could be depleted so rapidly as density increases that multilayer growth would be prevented.

A preliminary study was done, screening factors which might not limit monolayer growth but could be limiting for growth of multilayers. First, cells were seeded at a sub-optimal density, originally at 20,000 cells/cm² with DMEM+. A given factor was then added to the medium to test whether the number of surviving cells increased or if the apparent health of the cells improved as compared to the control plate without the factor.

The cultures were setup in polystyrene dishes, 35 mm diameter, incubated in a 5% CO₂-air incubator, and replenished every three days. After ten days, the plates were examined. A factor that had a beneficial effect on multilayer growth or cell health in three separate experiments was added to the medium used as the control in the next experiment in which another factor was tested using a somewhat lower seeding density.

These series of studies resulted in a novel growth medium in which DMEM+is supplemented with a number of factors. The factors are given in the order in which their beneficial effect was detected. The growth medium contains insulin 10⁻⁶M, epidermal growth factor 20 ng/ml, transferrin 5 mg/L, corticosterone 4×10⁴M, triiodothyronine 5×10⁻⁷M, vasopressin 5×10⁻⁹M, galactose 400 mg/L, 2-phosphoascorbate 0.2 mg/L, phosphoethanolamine 2×10⁻⁴M, putrescine 0.2 mg/L, Vitamin B 12 1 mg/L, biotin 1 mgm/L, Vitamin E dissolved in soybean oil 2×10⁻⁶M, ergocalciferel 2×10⁻⁸M, ergothioneine 1×10⁻⁶ M, acetyl carnitine 10⁻³ M, acetyl cysteine 10⁻⁸M, selenium 5×10⁻⁴M, ZnSO₄.7H₂O, 2×10⁻⁶M, CuSO₄.5H₂O, 2×10⁻⁷M, MnSO₄ 2×10⁻⁸M, testosterone 2×10⁻⁸M and progesterone 10×⁻⁷M. Some of these factors had previously been shown to improve growth of hep G2 cells, and are all normal components of plasma. Many of the factors, particularly the anti-oxidants, are present in serum at much higher concentrations than those found to be optimal.

### Example 9

Albumin Production by Hep G2 Cells in Polystyrene Plates With Unproved Nutrient Medium

The growth of Hep G2 cells seeded at 30,000 cells/cm² in COPS plates and polystyrene dishes in the novel improved medium was compared to growth of like cells seeded on polystyrene dishes in DMEM+. Although, the improved medium had been found to improve growth from low cell densities, it did not cause any significant increase in either the rate of growth or the final density of cells in the monolayers grown on the polystyrene plates as compared to the controls in DMEM+.

The cells that were seeded on the polystyrene plates with the improved nutrient medium secreted a higher level of albumin than the cells grown on the polystyrene plates with standard media. Importantly, the cells that were seeded on the COPS plate with improved nutrient media, secreted a much higher level of albumin than any of the cells seeded on the polystyrene plates with or without the improved nutrient media.

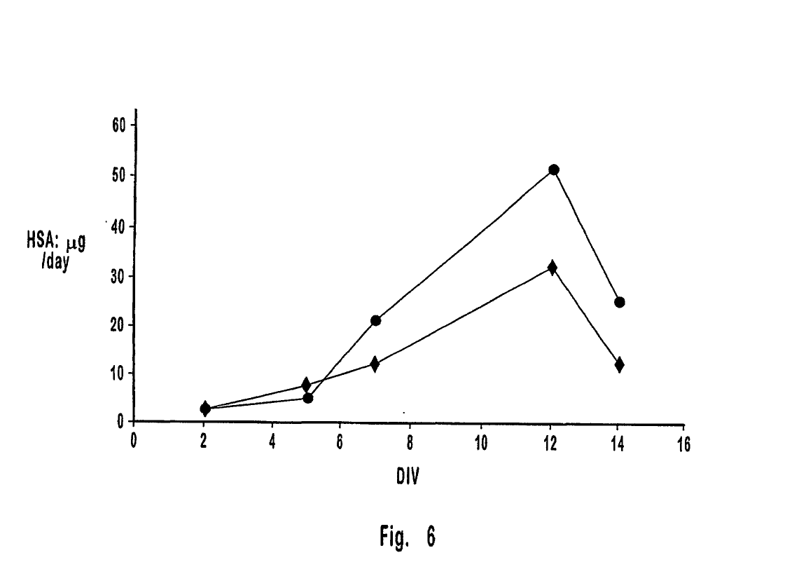

As shown in FIG. 6, the Hep G2 cells growing in a COPS plate with the improved nutrient medium secreted a higher level of albumin as compared to the cells grown in a COPS plate in DMEM+. These results show that the improved medium supports a more aerobic metabolism.

### Example 10

Cell Growth in COPS Plate With Improved Nutrient Medium

When cultured in COPS plates, in which cells had the benefits of improved oxygenation, the rate of growth and final cell density was significantly better in the novel medium than in DMEM+.

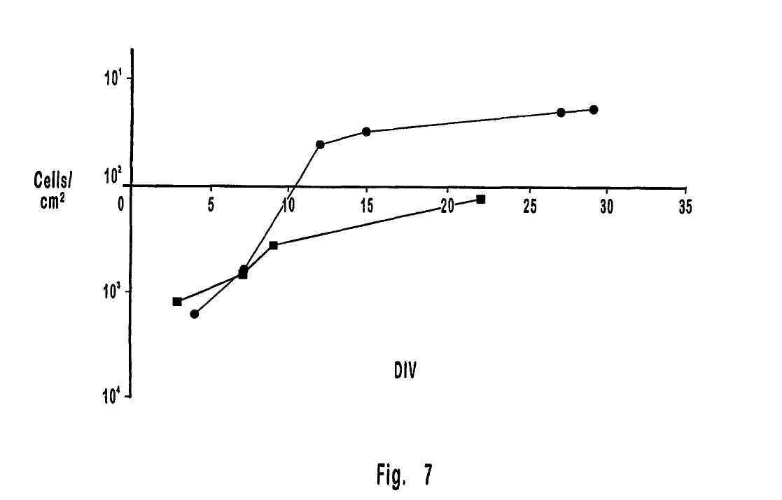

Cultures of hep G2 cells were seeded in parallel at 30,000 cells/cm², in the polystyrene tissue culture dishes and in COPS plates with collagen-coated FC-71 substrata. The plates were left open in an 5% CO₂-air incubator, so the cells were maintained with 20-21% oxygen and replenished every three days. Growth was followed overtime by assaying two wells from each series for increase in DNA. As shown in FIG. 7, the cells grew nearly three times faster in the COPS plate during the first twelve days than in the polystyrene dishes. In the dishes the growth rate declined markedly at about eight days upon the formation of a monolayer with a density of about 8×10⁻⁵ cells/cm². Although not indicated by the DNA data, the quality of the cells in the polystyrene dishes declined markedly after the monolayers had formed. In contrast, cells in the COPS plate with the novel supplemented medium grew rapidly for twelve days and continued to grow beyond the monolayer stage reaching densities nearly ten times higher than in the dish cultures.

The present invention maybe embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

1. A method for the attachment and growth of cells comprising the steps of:
contacting a surface with a perfluorocarbon;
mixing a perfluoro-aldehyde with the perfluorocarbon;
bonding an attachment factor to the perfluoro-aldehyde;
adding aqueous growth media, and
adding at least one anchorage-dependent cell and allowing the cell to grow.

2. The method of claim 1, wherein the perfluorocarbon is selected from the group consisting of perfluorotrihexylamine (FC-71), perfluorotripentylamine (FC-70), perfluorodecalin, and perfluorortributylamine.

3. The method of claim 1, wherein the perfluoro-aldehyde has at least 8 terminal perfluorinated carbons.

4. The method of claim 1, wherein the perfluoro-aldehyde has from about 8 to about 30 terminal perfluorinated carbons.

5. The method of claim 1, wherein the perfluoro-aldehyde has about 17 terminal perfluorinated carbons.

6. The method of claim 1, wherein the attachment factor is selected from the group consisting of gelatin, collagen, albumin, fibronectin, poly-1-lysine, and mixtures thereof.

7. The method of claim 6, wherein the attachment factor is poly-1-lysine and the method further comprises the step of coupling a rare matrix factor to the poly-1-lysine.

8. The method of claim 7, wherein the rare matrix factor is selected from the group consisting of laminin, enactin, proteoglycans, and a mixture thereof.

9. The method of claim 1, wherein the cells are eukaryotic.

10. The method of claim 9, wherein the cells are co-cultured.

11. The method of claim 10, wherein the eukaryotic cells comprise cells from an established cell line.

12. The method of claim 11, wherein the cells from an established cell line are cancer cells.

13. The method of claim 12, wherein the cancer cells are Hep G2 cells.

14. The method of claim 9, wherein the eukaryotic cells comprise primary cells.

15. The method of claim 14, wherein the primary cells are selected from the group consisting of hepatocytes, liver cells, kidney cells, brain cells, bone marrow cells, nerve cells, heart cells, spleen cells, stem cells and co-cultures of the above.

16. The method of claim 9, wherein the cells grow to form multiple cell layers.

17. The method of claim 1, wherein the aqueous growth media is not in direct contact with the perfluorocarbon.

18. The method of claim 1, wherein the surface is exposed to a defined gas mixture in which the level of oxygen differs from ambient levels.

19. The method of claim 1, wherein the surface is an open system.

20. The method of claim 1, wherein the aqueous growth media comprises Dulbecco's modified Eagles's medium, epidermal growth factor, pyruvate, insulin, transferrin, progesterone, corticosterone, triiodthyronine, vasopressin, galactose, 2-phosphoascorbate, phosphoethanolamine, putrescine, Vitamin B 12, biotin, Vitamin E, ergocalciferol, ergothioneine, acetyl carnitine, acetyl cysteine, selenium, ZnSO₄.7H₂O, CuSO₄.5H₂O, MnSO₄, and testosterone.

21. A culture vessel for growing anchorage-dependent cells comprising:
a shell enclosing a chamber;
an inlet port and an outlet port for gas exchange, the ports being operably disposed in relation to the shell; and
at least one well for cell growth disposed within the chamber, the at least one well comprising at least one well wall and a bottom wherein the well wall is constructed of an oxygen permeable material.

22. The culture vessel of claim 21, wherein the well wall is made of silicon.

23. The culture vessel of claim 22, wherein the silicone is selected from the group consisting of platinum-treated silicon and peroxide treated silicon.

24. The culture vessel of claim 21, wherein the well wall is constructed of silicone tubing.

25. The culture vessel of claim 21, wherein the bottom is made from an optically clear material.

26. The culture vessel of claim 21, wherein the inlet and outlet ports are left open.

27. The culture vessel of claim 21, wherein the inlet and outlet ports are attached to a ventilation system to maintain a desired oxygen level within the chamber.

28. A method for the attachment and growth of cells comprising the steps of:
obtaining a culture vessel comprising a shell enclosing a chamber, an inlet port and an outlet port for gas exchange that are operably disposed in relation to the shell, and at least one well for cell growth disposed within the chamber, the well comprising at least one well wall constructed of an oxygen permeable material and a bottom;
contacting the well with a perfluorocarbon;
mixing a perfluoro-aldehyde with the perfluorocarbon;
bonding an attachment factor to the perfluoro-aldehyde;
adding aqueous growth media; and
adding at least one anchorage-dependent cell and allowing the cell to grow.

29. The method of claim 28, wherein the perfluorocarbon is selected from the group consisting of perfluorotrihexylamine (FC-71), perfluorotripentylamine (C-70), perfluorodecalin, perfluorortributylamine, and.

30. The method of claim 28, wherein the perfluoro-aldehyde has at least 8 terminal perfluorinated carbons.

31. The method of claim 28, wherein the attachment factor is selected from the group consisting of gelatin, collagen, albumin, fibronectin, and poly-1-lysine.

32. The method of claim 31, further comprising the step of coupling a rare matrix actor to the poly-1-lysine.

33. The method of claim 28, wherein the aqueous growth media is not in direct contact with the perfluorocarbon.

34. The method of claim 28, wherein the aqueous growthmediacomprisesDulbecco's modified Eagles's medium, epidermal growth factor, pyruvate, insulin, transferrin, progesterone, corticosterone, triiodthyronine, vasopressin, galactose, 2-phosphoascorbate, phosphoethanolamine, putrescine, Vitamin B 12, biotin, Vitamin E, ergocalciferol, ergothioneine, acetyl carnitine, acetyl cysteine, selenium, ZnSO₄.7H₂O, CuSO₄.5H₂O, MnSO₄, and testosterone.

35. The method of claim 28, wherein the well wall is made of silicon.

36. The method of claim 35, wherein the silicone is platinum-treated.

37. The method of claim 28, wherein the bottom is made from an optically clear material.

38. The method of claim 28, wherein the inlet and outlet ports are left open.

39. The method of claim 28, wherein the inlet and outlet ports are attached to a ventilation system to maintain a desired oxygen level within the chamber.
